(19) United States
(12) Patent Application Publication
(10) Pub. No.: US 2026/0000671 A1
(43) Pub. Date: Jan. 1, 2026

(54) CONCENTRATED SOLUTIONS COMPRISING 5,10-METHYLENE-(6R)-TETRAHYDROFOLIC ACID

(71) Applicant: Merck Patent GMBH, Darmstadt (DE)

(72) Inventors: Rudolf Moser, Schaffhausen (CH); Viola Groehn, Dachsen (CH); Thomas Ammann, Marthalen (CH); Jean-Pierre Knapp, Schaffhausen (CH); Marianne Svaerd, Veberoed (SE)

(21) Appl. No.: 18/869,144

(22) PCT Filed: Jun. 5, 2023
(86) PCT No.: PCT/EP2023/064971

§ 371 Date: Nov. 25, 2024

(30) Foreign Application Priority Data
Jun. 8, 2022 (EP) 22177931.7

(51) Int. Cl. A61K 31/519; A61K 9/00; A61K 9/08

(52) U.S. Cl. CPC A61K 31/519; A61K 9/0019; A61K 9/08

(57) ABSTRACT

The present invention relates to stable high-content solutions comprising 5,10-methylene-(6R)-tetrahydrofolic acid (5,10-CH₂-(6R)-THF), which solutions are free of extraneous stabilizing agents.

The present invention relates to liquid compositions comprising a high content of 5,10-methylene-(6R)-tetrahydrofolic acid, which compositions do not contain any extraneous stabilizers.

### BACKGROUND OF THE INVENTION

5,10-methylenetetrahydrofolic acid is known as a medicament used in combination with 5-fluorouracil (5-FU) in the treatment of solid tumors (Seley, K. L. Drugs 4 (1), 99, 2001). The active isomeric form 5,10-methylene-(6R)-tetrahydrofolic acid (referred to as 5,10-CH₂-(6R)-THF in the following), achieves its chemotherapeutic effect together with the base analogue and 5-FU metabolite 5-FdUMP by inhibiting the enzyme thymidylate synthase (TS). TS catalyzes the conversion of deoxyuridylate (dUMP) to deoxythymidylate (dTMP), which is an essential building block for DNA synthesis. Deactivation of TS occurs by formation of a covalent, ternary inhibition complex between TS, the base analogue 5-FdUMP, which is a metabolite of 5-FU, and 5,10-CH₂-(6R)-THF.

An enhancement of the cytotoxic effect of 5-FU can be achieved by increasing the intracellular concentration of 5,10-CH₂-(6R)-THF, whereupon the stability of the ternary inhibition complex is increased. This causes direct inhibition of DNA synthesis and repair, which ultimately results in cell death and delay of tumor growth. In order to achieve high intracellular concentrations of 5,10-CH₂-(6R)-THF, the application of respective high content products is desired.

However, there are undesirable properties associated with 5,10-CH₂-(6R)-THF that limit its pharmaceutical use. For example, 5,10-CH₂-(6R)-THF is highly susceptible to oxidation and chemical degradation that results in unfavourably high impurity levels.

Susceptibility to oxidation and chemical degradation of 5,10-CH₂-(6R)-THF is especially high in aqueous solution, or when the compound is present in its amorphous form where it has a large surface (e.g. in its pharmaceutical use form as a lyophilisate), or in re-dissolved form such as solutions for injection. It is well known that to be amenable for pharmaceutical use, the respective composition needs to fulfil several requirements including high chemical and isomeric stability, such that effective storage over an acceptable period of time can be achieved, without exhibiting a significant change in the composition's physicochemical characteristics, ease of handling and processing, etc.

5,10-methylenetetrahydrofolic acid is an addition product of tetrahydrofolic acid and form-aldehyde (see e.g. Poe, M. et al. Biochemistry 18 (24), 5527, 1979; Kallen, R. G. Methods in Enzymology 18B, 705, 1971) and is known for its extremely high sensitivity to oxidation by air as well as instability in neutral and/or acidic environments potentially leading to chemical degradation and/or hydrolysis (see e.g. Odin, E. et al., Cancer Investigation 16 (7), 447, 1998; Osborn, M. J. et al., J. Am. Chem. Soc. 82, 4921, 1960; Hawkes, J., and Villota, R. Food Sci. Nutr. 28, 439, 1989). Attempts to stabilize compositions of 5,10-methylenetetrahydrofolates included e.g. (i) rigorous exclusion of atmospheric oxygen by the use of special technical devices for the reconstitution of solid formulations and the injection of 5,10-methylenetetrahydrofolates in an air-free environment (see e.g. Odin, E. et al., Cancer Investigation 16 (7), 447, 1998; U.S. Pat. No. 4,564,054); (ii) addition of a reducing agent such as L(+)-ascorbic acid or salts thereof, reduced gamma-glutathione, beta-mercaptoethanol, thioglycerol, N-acetyl-L-cysteine, etc. as an antioxidant for the highly sensitive 5,10-methylenetetrahydrofolic acid and for tetrahydrofolic acid in particular; (iii) stabilization by means of cyclodextrin inclusion compounds (see e.g. EP 0 579 996); (iv) addition of citrate while adjusting the pH to a basic value (see e.g. EP 1 641 460); or (v) formation of various crystalline forms such as the sulfate salts (see e.g. EP 0 537 492) or hemisulfate salts (see e.g. EP 2 837 631).

It has previously been found (see e.g. WO 2019/034673) that the addition of a dicarboxylic acid to 5,10-CH₂-(6R)-THF helps to maintain the purity of the active ingredient 5,10-CH₂-(6R)-THF on a remarkably high level during the lyophilization process and at the same time maintain the amounts of by-products at an acceptable low level. The addition of reducing agents such as L-(+)-ascorbic acid has also been found to have a positive influence on the stability of 5,10-CH₂-(6R)-THF.

The addition of citrate while adjusting the pH to a basic value (see e.g. EP 1 641 460) in particular has this stabilizing effect, which is described at great length in U.S. Pat. No. 10,059,710 B2. Thus, the stabilization of 5,10-CH₂-(6R)-THF and of its hemisulfate salt with citrate at slightly acidic to basic pH values is described to be due to a synergistic effect of the citrate buffer solution in this pH range.

It should be mentioned that U.S. Pat. No. 10,059,710 B2 also mentions citrate-free formulations of 5,10-CH₂-(6R)-THF, without, however, offering guidance on the preparation of such formulations. On the contrary, U.S. Pat. No. 10,059,710 B2 teaches the skilled person that citrate (or other buffers) is required for preparing stable compositions of 5,10-CH₂-(6R)-THF.

From a clinical perspective the availability of stable solutions and lyophilisates of 5,10-CH₂-(6R)-THF having a high content of the active ingredient and being free of any kind of stabilizers would be an advantage. Citric acid, for example, has been linked to various undesired effects like e.g. QTc elongation (Laspina et al. Transfusion 42 (2002) p. 899, Toyoshima et al. Clinical Nutrition (2006) 25, 653-660), inducing hypocalcaemia (Payne et. Al. J. Physiol. (1964), 170, pp. 613-620), etc.

The teachings of the prior art, including U.S. Pat. No. 10,059,710 B2 (supra), does not readily allow the preparation of such stabilizer-free compositions of 5,10-CH₂-(6R)-THF, and thus there still remains a great need for stable liquid and solid-state pharmaceutical compositions of 5,10-CH₂-(6R)-THF, which do not contain extraneous stabilizing compounds such as stabilizers, buffers, reducing agents and the like.

It is a derived problem that the stabilized versions of 5,10-CH₂-(6R)-THF known in the prior art usually contain less than 50% of the active drug compound due to the dilution in the final dosage form of the drug by the stabilizing additives. As an example, the company Adventrx Pharmaceuticals carried out stability studies on their drug candidate CoFactor®, i.e. the calcium salt of the diastereomer mixture 5,10-methylene-(6R,S)-tetrahydrofolic acid, which were disclosed i.a. in WO 2007/064968. The chemical stability of the diastereomer mixture 5,10-methylene-(6R,S)-tetrahydrofolic acid is assumed to be similar to the pure diastereomer 5,10-CH₂-(6R)-THF of the present invention. The study compared the stability of nonformulated 5,10-methylene-(6R,S)-tetrahydrofolic acid with 5,10-methylene-(6R,S)-tetrahydrofolic acid formulated with trisodium citrate alone or formulated with both ascorbic acid and trisodium citrate; both of which compounds are well-known anti-oxidants (see FIG. 1).

Linear regression analysis of the stability profiles of the isolated lyophilizates showed that 5,10-methylene-(6R,S)-tetrahydrofolic acid degradation was linear over time (see FIG. 2). The degradation rate (slope of the best-fit line) for each formulation (re-constituted lyophilizate) showed the following order, from fastest to slowest degradation rate: nonformulated>formulated with only trisodium citrate>formulated with both ascorbic acid and trisodium citrate (FIG. 2). Nonformulated 5,10-methylene-(6R,S)-tetrahydrofolic acid was thus found to lose 2.3% purity per hour, resulting in a purity of 84% after 7 hours, whereas formulations containing trisodium citrate+ascorbic acid had much higher stability, resulting in a purity of about 95% after 7 hours.

However, the solutions disclosed in WO 2007/064968 for the purpose of preparing the most stable lyophilizates contain less than 5% by weight 5,10-methylene-(6R,S)-tetrahydrofolic acid, and the resulting lyophilizates contain less than 20% by weight 5,10-methylene-(6R,S)-tetrahydrofolic acid (see FIG. 3).

Additionally, known stabilizers such as citric acid, for example, have been linked to various undesired effects like e.g. QTc elongation (Laspina et al. Transfusion 42 (2002) p. 899, Toyoshima et al. Clinical Nutrition (2006) 25, 653-660), inducing hypocalcaemia (Payne et. Al. J. Physiol. (1964), 170, pp. 613-620), etc. From a clinical perspective the availability of stable solutions and lyophilizates of 5,10-CH₂-(6R)-THF having a high content of the active ingredient and being free of any kind of stabilizers would be an advantage.

There thus still remains a great need for stable pharmaceutical compositions having a high content of 5,10-methylene-(6R)-tetrahydrofolic acid.

### SUMMARY OF THE INVENTION

It has now surprisingly been found that highly concentrated, stable solutions, having a pH between about 8 and 9.5, comprising 5,10-methylene-(6R)-tetrahydrofolic acid (5,10-CH₂-(6R)-THF), can be prepared from 5,10-CH₂-(6R)-THF or a salt thereof, in the absence of extraneous stabilizing compounds such as stabilizers, buffers, reducing agents and the like.

The concentrated solutions and lyophilisates of the present invention thus overcome the previously discussed known drawbacks and allow for the preparation of liquid and solid-state pharmaceutical compositions of high purity and a low content of oxidation products and other chemical degradation products. The advantageous stability characteristics of the solutions of the present invention will allow the effective, and safe use of 5,10-CH₂-(6R)-THF in medicinal applications.

In a first aspect the present invention relates to a concentrated aqueous solution comprising 5,10-methylene-(6R)-tetrahydrofolic acid (5,10-CH₂-(6R)-THF), which concentrated solution further does not contain any stabilizing agents such as stabilizers, buffers, reducing agents and the like, as defined hereinbelow.

In a second aspect the present invention further relates to a concentrated aqueous solution according to the first aspect, or a reconstituted or diluted aqueous solution thereof, for use in the treatment of cancer in human patient.

In a third aspect the present invention further relates to a method of treatment of cancer in human patients comprising administering a concentrated aqueous solution according to the first aspect, or a reconstituted or diluted aqueous solution thereof, to a human patient in need thereof.

In a fourth aspect the present invention further relates to a composition comprising 5,10-CH₂-(6R)-THF obtainable by reconstitution or dilution of a concentrated aqueous solution according to the first aspect.

### BRIEF DESCRIPTION OF THE FIGURES

FIG. 1 is adapted from Table 2 in WO 2007/064968 and demonstrates the stability over time of non-formulated and various formulated forms of 5,10-methylene-(6R,S)-tetrahydrofolic acid (% normalized purity). As can be seen, each formulation had a different stability profile. Thus, nonformulated 5,10-methylene-(6R,S)-tetrahydrofolic acid at neutral pH degraded rapidly over time. 24 hours following dissolution in water, the purity of nonformulated 5,10-methylene-(6R,S)-tetrahydrofolic acid was only 44.9% of the starting purity. The reference formulation formulated only with trisodium citrate (pH adjusted>7.5) showed slower degradation following dissolution in water.

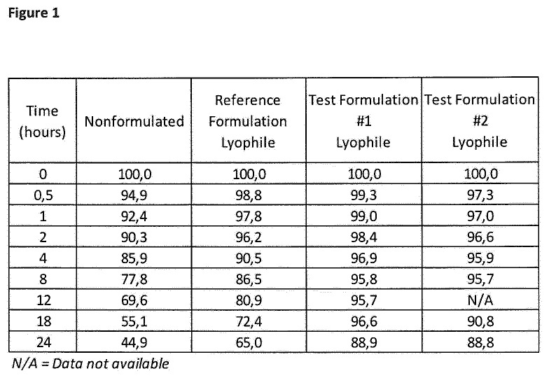

However, purity after 24 hours was still only 65% compared to the starting purity, indicating degradation was not efficiently inhibited by the addition of trisodium citrate and adjustment of pH. The two test formulations #1 and #2 (i.e. 5,10-methylene-(6R,S)-tetrahydrofolic acid formulated with both ascorbic acid and trisodium citrate) were the most stable formulations.

FIG. 2 is adapted from FIG. 1 in WO 2007/064968 and demonstrates graphically the tabulated results of FIG. 1 herein.

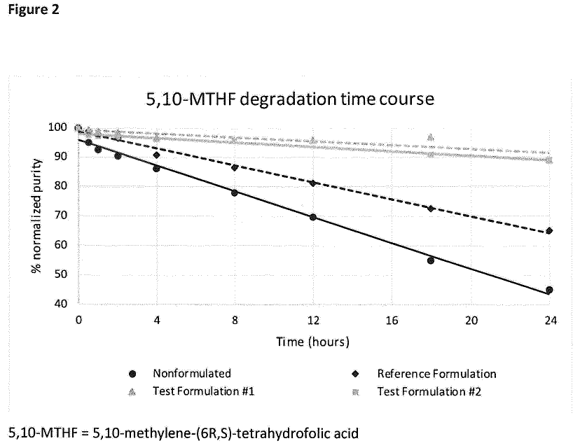

FIG. 3 is a table adapted from Example 1 of WO 2007/064968 showing the composition of the non-formulated and formulated forms of 5,10-methylene-(6R,S)-tetrahydrofolic acid shown in FIG. 1 and FIG. 2 herein.

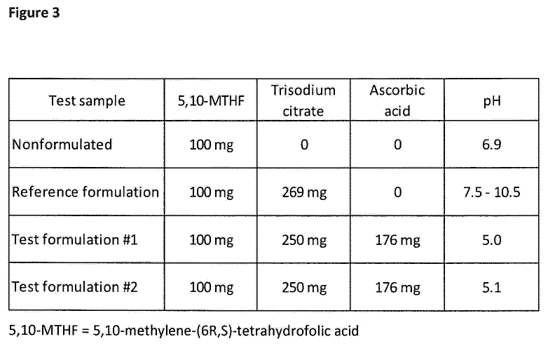

FIG. 4 shows the purity analyses of four identical solutions of sodium salt of 5,10-methylene-(6R)-tetrahydrofolic acid of the present invention tested at four different conditions: 5° C. without a blanket of N₂, 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown for a total period of 7 hours. As can be seen from the graphs, the solutions are very stable under the storage conditions, changing from an initial purity between 96.6-97% to a purity of 96.4-96.5% (area %). As can also be seen, the effect of N₂ blanketing is minimal.

FIG. 5 shows analyses of the same four solutions of sodium salt of 5,10-methylene-(6R)-tetrahydrofolic acid as shown in FIG. 4 herein. In FIG. 5, the development over 7 hours of the main impurity, 10-formyl-(6R)-tetrahydrofolic acid (10-FTHFA) in the solutions as produced in Example 3 when stored at 2-8° C. is shown. As can be seen, the level of this impurity is practically constant over time.

### DEFINITIONS

In the present text, the term “stabilizers” or “stabilizing agents” relates to buffers such as citrate (or citric acid and salts thereof); dicarboxylates such as succinate, malate and maleate; tris(hydroxymethyl)aminomethane (TRIS); N-tris(hydroxymethyl)methyl-2-aminoethane-sulfonic acid (TES); 3-(N-morpholino) propanesulfonic acid (MOPS); N,N-bis(2-hydroxyethyl)-2-aminoethanesulfonic acid (BES); MES; MOPSO; HEPES; phosphate; carbonate; ammonium; mono-, di-, and tri-alkylammonium; mono-, di-, and tri-hydroxylalkylammonium; glutamate; borate; lactate, as well as combinations of these. The term “stabilizers” or “stabilizing agents” further relates to reducing agents such as L-(+) ascorbic acid or salts thereof, reduced γ-glutathione, β-mercaptoethanol, thioglycerol, N-acetyl-L-cysteine, etc. which may act as an antioxidant for the sensitive 5,10-methylenetetrahydrofolic acid, and for the tetrahydrofolic acid in particular.

In the present text, the term “buffer” relates to citrate (or citric acid and salts thereof), dicarboxylates such as succinate, malate and maleate; tris(hydroxymethyl)aminomethane (TRIS); N-tris(hydroxymethyl)methyl-2-aminoethanesulfonic acid (TES); 3-(N-morpholino) propanesulfonic acid (MOPS); N,N-bis(2-hydroxyethyl)-2-aminoethane-sulfonic acid (BES); MES; MOPSO; HEPES; phosphate; carbonate; ammonium; mono-, di- and tri-alkylammonium; mono-, di- and tri-hydroxylalkylammonium; glutamate; borate; lactate, as well as combinations of these.

In the present text, the term “reducing agent” relates to L-(+) ascorbic acid or salts thereof, reduced γ-glutathione, β-mercaptoethanol, thioglycerol and N-acetyl-L-cysteine.

In the present text, the term “solvent” relates to solvents which may be used in freeze drying processes. “Solutions” as referred to in the present text, comprise aqueous solutions as well as solutions in organic solvents. Typically, “aqueous solutions” mean solutions in water, saline solutions, water containing small amounts of buffers, water containing isotonic amounts of NaCl, or mixtures of water with organic solvents, and the like. Typical organic solvents include DMSO, acetonitrile, acetone, methanol, or ethanol.

### DETAILED DESCRIPTION OF THE INVENTION

It has now surprisingly been found that highly concentrated solutions of 5,10-CH₂-(6R)-THF in either pure water or saline can be prepared, which have a similar stability towards hydrolysis and oxidation as the previously described solutions containing various stabilizers as described above (see e.g. WO2019034673, US 2007/0099866 and U.S. Pat. No. 10,059,710 B2). The solutions of the present invention, which are free of stabilizers (in the following referred to as “stabilizer-free”), contain more than 40 mg 5,10-CH₂-(6R)-THF per ml, such as more than 45 mg/ml, more than 55 mg/ml, more than 60 mg/ml, more than 65 mg/ml, such as preferably 70 mg 5,10-CH₂-(6R)-THF per ml. Solutions of higher concentration can be prepared but become very viscous.

The highly concentrated solutions according to the instant invention remain stable for at least 8 days at 5±3° C. For longer term storage, the aqueous solutions can be filled in containers and freeze-dried (lyophilized) to a stable, non-sticky, stabilizer-free powder. The powder can be reconstituted with a diluent to a set concentration for administration. Alternatively, aqueous solutions can be produced in a “ready to use” concentration and filled in containers, e.g. vials or ampoules. Such solutions, or reconstituted lyophilisates, can be administered either intramuscularly or intravenously.

The solutions of the invention may contain additional excipients. For example, electrolytes, sugars and/or polyols such as dextrose, glycerol, mannitol and sodium chloride may be added to adjust the osmolality. Osmolality adjustment can be done before or after preparing the solutions for administration. The reconstituted (diluted) solutions preferably have an osmolality in the range of 250-350 mOsm. However, an osmolality of 200-600 mOsm can be tolerated as well and will depend on the volume to be administered as well as on the injection/infusion time.

The pH of the solutions is typically in the range of 8.0 to 9.0, preferably in the range of 8.4 to 8.8 and can be adjusted during drug product manufacturing with e.g. small amounts of hydro-chloric acid or sodium hydroxide.

The high stability obtained for the concentrated solutions of 5,10-CH₂-(6R)-THF at pH 8.6 is highly surprising in view of the prior art described above, in which the presence of a stabilizer like citrate and/or ascorbic acid would have been mandatory.

The first aspect of the present invention thus relates to a concentrated aqueous solution comprising 5,10-methylene-(6R)-tetrahydrofolic acid and does not contain stabilizers.

In a preferred embodiment the present invention relates to a concentrated solution of 5,10-CH₂-(6R)-THF containing more than 45 mg 5,10-CH₂-(6R)-THF per ml, such as more than 50 mg/ml, more than 55 mg/ml, more than 60 mg/ml, more than 65 mg/ml, such as preferably 70 mg 5,10-CH₂-(6R)-THF per ml, which solution further does not contain any stabilizing agents such as stabilizers, buffers, reducing agents and the like, as defined hereinbelow.

In a preferred embodiment, the composition according to the first aspect is prepared from 5,10-CH₂-(6R)-THF hemisulfate.

In another embodiment, the composition according to the first aspect does not contain a compound selected from the group consisting of citrate (or citric acid and salts thereof); tris(hydroxymethyl)aminomethane (TRIS); N-tris(hydroxymethyl)methyl-2-aminoethanesulfonic acid (TES); 3-(N-morpholino) propanesulfonic acid (MOPS); N,N-bis(2-hydroxy-ethyl)-2-aminoethanesulfonic acid (BES); MES; MOPSO; HEPES; phosphate; acetate; succinate; carbonate; ammonium; mono-, di-, and trialkylammonium; mono-, di-, and tri-hydroxylalkylammonium; maleate; glutamate; borate; lactate; L-(+) ascorbic acid or salts thereof; reduced γ-glutathione; β-mercaptoethanol; thioglycerol; N-acetyl-L-cysteine, and combinations of these.

In a second aspect the present invention further relates to a concentrated solution according to the first aspect, or reconstituted or diluted aqueous solutions thereof, for use in the treatment of cancer in human patient.

In a third aspect the present invention further relates to a method of treatment of cancer in human patients comprising administering a concentrated solution according to the first aspect, or a reconstituted or diluted aqueous solution thereof, to a human patient in need thereof.

One embodiment is directed to a concentrated solution comprising 5,10-CH₂-(6R)-THF, which does not contain tricarboxylic acids, or salts thereof.

One embodiment is directed to a concentrated solution comprising 5,10-CH₂-(6R)-THF, which does not contain dicarboxylic acids, or salts thereof.

One embodiment is directed to a concentrated solution comprising 5,10-CH₂-(6R)-THF, which does not contain dicarboxylic acids, or salts thereof, or reducing agents such as L-(+) ascorbic acid.

One embodiment is directed to a concentrated solution comprising 5,10-CH₂-(6R)-THF having a purity of greater than 98%.

One embodiment is directed to a stabilizer-free concentrated solution comprising 5,10-CH₂-(6R)-THF.

The concentrated solutions of the present invention are preferably reconstituted (diluted) into an aqueous pharmaceutical formulation to be administered into a patient in need thereof.

A further aspect is directed to a process for the preparation of concentrated aqueous solutions of the present invention, which comprises the steps of:
(i) dissolving 45-70 mg/ml 5,10-methylene-(6R)-tetrahydrofolic acid, or a salt thereof, in water, or optionally saline, such as 0.4-0.95% % aqueous NaCl, such as 0.45% NaCl or 0.9% NaCl;
(ii) optionally adding excipients i.a. to adjust the osmolality of the solution.

In an embodiment, the salt of 5,10-methylene-(6R)-tetrahydrofolic acid used in step (i) is the hemisulfate or the sulfate salt of 5,10-methylene-(6R)-tetrahydrofolic acid.

Optionally an aqueous base, like NaOH or KOH is added in step (i) to complete dissolution of the 5,10-methylene-(6R)-tetrahydrofolic acid compound.

The solution of step (i) can optionally be filtered through a sterile filter, optionally under an inert atmosphere before step (ii) is performed.

In separate embodiments, the pH of the solution in step (i) is above 6, usually about 8-14, preferably about 9-10 or 8.5-9.5.

In a fourth aspect the present invention further relates to a composition comprising 5,10-CH₂-(6R)-THF obtainable by reconstitution or dilution of a composition according to the first aspect. Adjustment of osmolality can be done (if needed) at the time when the lyophilisate is dissolved (reconstituted).

A further aspect is directed to reconstituted pharmaceutical concentrated solutions of the present invention comprising 5,10-CH₂-(6R)-THF and a pharmaceutically acceptable carrier or diluent, such as sterile water or a liquid pharmaceutically acceptable vehicle, optionally further comprising at least one additional therapeutic agent including but not limited to, bactericides, antibiotics, antivirals, antiseptics, antineoplastics, anticancer compounds such as chemotherapeutic agents, antifungals, and/or anti-inflammatory agents or other bioactive or therapeutic agents that are suitable for human use, in particular anticancer compounds such as chemotherapeutic agents, for example 5-FU (5-fluorouracil) and derivatives thereof, and antifolates, e.g. methotrexate, Pemetrexed.

“Liquid pharmaceutically acceptable vehicle” refers to propylene glycol, a polyethylene glycol, ethanol, dimethyl sulfoxide (DMSO), N-methylpyrrolidinone (NMP), glycofurol, isopropylidene glycerol (Solketal), glycerol formal, acetone, tetrahydrofurfuryl alcohol, monoglyme, diglyme, dimethyl isosorbide or ethyl lactate, and mixtures, incl. aqueous mixtures thereof.

A further aspect is directed to the use of the reconstituted concentrated solutions comprising 5,10-CH₂-(6R)-THF of the present invention in therapy, preferably in cancer chemotherapy.

Yet another aspect is directed at the reconstituted concentrated solutions comprising 5,10-CH₂-(6R)-THF according to the present invention for use in the treatment of cancer, preferably in human patients.

In a preferred embodiment, the reconstituted concentrated solutions comprising 5,10-CH₂-(6R)-THF according to the present invention are administered in combination with 5-fluorouracil (5-FU) or in a dosing regimen further comprising the administration of fluorouracil (5-FU).

Further aspects of the present invention are directed at the use of the concentrated solutions comprising 5,10-CH₂-(6R)-THF of the present invention, including reconstituted (diluted) solutions thereof, in combination with at least one or more additional therapeutic agents. Preferred additional therapeutic agents comprise in this context chemotherapeutic agents and other anti-cancer drugs. Particularly preferred drugs, which may be combined with the pharmaceutical compositions of the present invention, comprise fluorinated nucleic acids such as 5-fluorouracil or prodrugs or analogues thereof.

### EXAMPLES

### HPLC

For the measurement of purity/content and degradation products an HPLC-UV Gradient Method was used: Column type: ODS, Mobile phase: A: aqueous Buffer; Mobile Phase: B: aqueous Buffer/Methanol, Run time: 30 min, Sample Solvent: aqueous Buffer.

### Water Content

The determination of the water content was made according to Ph. Eur. 2.5.32/USP<921/Method Ic>.

### Osmolality

The determination of the osmolality was made according to Ph. Eur. 2.2.35 (osmometer)/USP<785>.

### Example 1 Preparation of a Concentrated Solution of 5,10-CH₂-(6R)-THF in 0.9% NaCl

The description below is for preparation of 50 ml of 70 mg/ml 5,10-CH₂-(6R)-THF in saline solution. Example 2 below concerns the similar preparation of a concentrated solution in pure water. The preparation procedures can be adapted to any suitable volumes as needed.

The preparation procedures should not take more than 30 minutes. 5,10-CH₂-(6R)-THF solutions should always be prepared using cold solutions (2-8° C.) and the solutions must also be kept cold during mixing, by keeping the reaction vessel chilled. The temperature in the solutions should not be above 8° C. After the preparation is done, transfer the 5,10-CH₂-(6R)-THF solution directly to the refrigerator/cold box until the contemplated use as such, or until the solution is about to be lyophilized.

The saline solution procedure starts by preparing the following solutions:

| | |
|---|---|
| Solution A | 7.26 g 2M NaOH |
| | Add 0.9% NaCl solution up to 100 ml |
| Solution B | Mix 6 parts of solution A with 1 part |
| | of NaOH solution 32% |

Add 3750 mg of 5,10-CH₂-(6R)-THF hemisulfate to a volumetric flask or a container which allows for good mixing of 50 ml. Add 25 ml of Solution A to the flask with efficient stirring under an inert atmosphere. Adjust the pH slowly and carefully to 8.6±0.20 using Solution B. The pH should not exceed pH 9.2. Adjustment of the pH to 8.6±0.20 can be done using 0.1M HCl, if necessary.

Adjust the total volume to 50 ml with 0.9% NaCl, and measure the pH, which should be 8.6±0.20. Perform a sterile filtration using a 0.22 μm filter. The filtration should be performed using a hydrophilic PVDF type e.g. Millipore Durapore® 0.22 μm. The resulting solution contains 70 mg/ml 5,10-CH₂-(6R)-THF, pH 8.6±0.20, in saline solution.

### Example 2 Preparation of a Concentrated Solution of 5,10-CH₂-(6R)-THF in Water

See Example 1 above for general instructions.

The pure water solution procedure starts by preparing the following solutions:

| | |
|---|---|
| Solution A | 7.26 g 2M NaOH |
| | Add sterile filtered water up to 100 ml |
| Solution B | Mix 6 parts of solution A with 1 part of |
| | NaOH solution 32% |

Add 3750 mg of 5,10-CH₂-(6R)-THF hemisulfate to a volumetric flask or a container which allows for good mixing of 50 ml. Add 25 ml of Solution A to the flask with efficient stirring under an inert atmosphere. Adjust the pH slowly and carefully to 8.6±0.20 using Solution B. Adjustment of the pH to 8.6±0.20 can be done using 0.1M HCl, if necessary.

Adjust the total volume to 50 ml with sterile filtered water, and measure the pH, which should be 8.6±0.20. Perform a sterile filtration using a 0.22 μm filter. The filtration should be performed using a hydrophilic PVDF type e.g. Millipore Durapore® 0.22 μm. The resulting solution contains 70 mg/ml 5,10-CH₂-(6R)-THF, pH 8.6±0.20.

### Example 3: Stability Testing

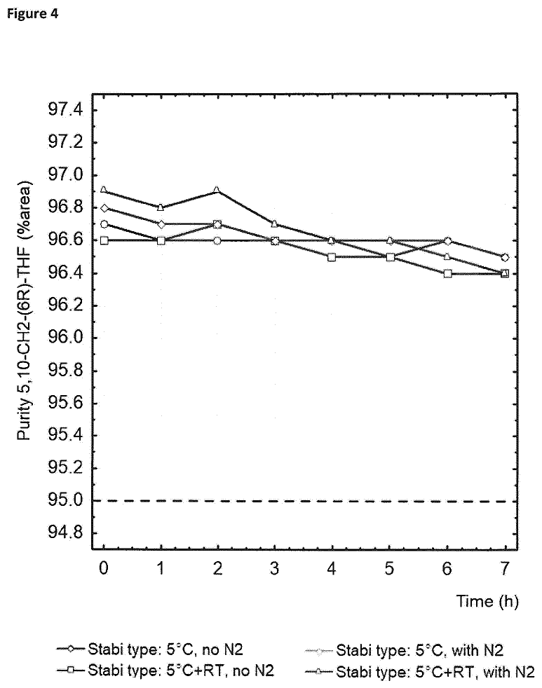

The solutions as produced in Example 2 were tested for stability under four different conditions: 7 hrs at 5° C. without a blanket of N₂, 7 hrs at 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown in FIG. 4. As can be seen from the graphs, the solutions are very stable under the storage conditions, changing form an initial purity between 96.6-97% to a purity of 96.4-96.5% (area %). As can also be seen from FIG. 4, the effect of N₂ blanketing on stability is minimal.

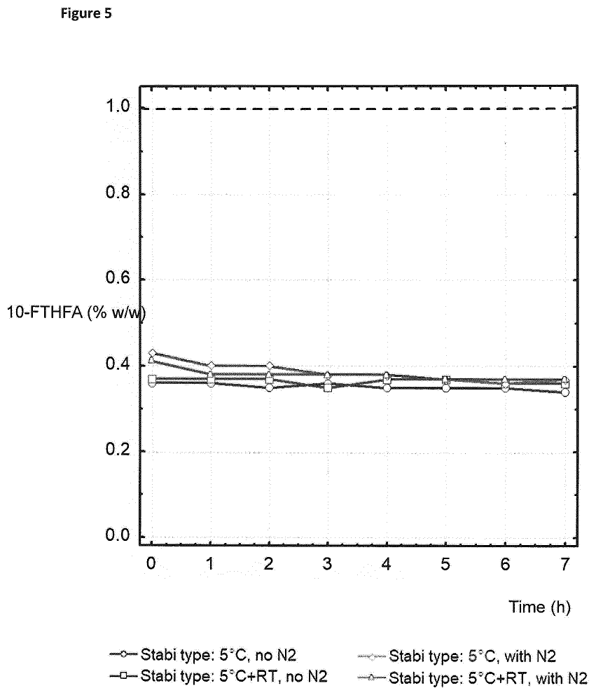

As part of the stability analysis, the development over 7 hours of the main impurity, 10-formyl-(6R)-tetrahydrofolic acid in the solutions as produced in Example 2 when stored at 2-8° C. was also measured (see FIG. 5). As can be seen, the level of this impurity is practically constant.

### Example 4 Preparation of a Stabilizer-Free Lyophilisate

If required, the concentrated solutions of Example 1 or 2 may be converted to stable lyophilizates, as follows: Fill the filtered solution from Example 1 or Example 2 at a temperature of 2-8° C. into vials (2 ml or 150 mg 5,10-CH₂-(6R)-THF per vial) while keeping the solution as cold as possible.

Freeze-dry the vials and seal them under a slight vacuum with nitrogen in the headspace. Crimp the vials. The resulting lyophilisate contains 70-80% w/w 5,10-CH₂-(6R)-THF.

1. A concentrated aqueous solution comprising 5,10-methylene-(6R)-tetrahydrofolic acid and does not contain stabilizers.

2. The concentrated aqueous solution according to claim 1, prepared from 5,10-methylene-(6R)-tetrahydrofolic acid hemisulfate.

3. The concentrated aqueous solution according to claim 1, wherein said stabilizers comprise the group consisting of citrate (or citric acid and salts thereof); tris(hydroxymethyl)aminomethane (TRIS); N-tris(hydroxymethyl)methyl-2-aminoethanesulfonic acid, (TES); 3-(N-morpholino) propanesulfonic acid (MOPS); N,N-bis(2-hydroxy-ethyl)-2-aminoethanesulfonic acid (BES); MES; MOPSO; HEPES; phosphate; acetate; succinate; carbonate; ammonium; mono-, di- and trialkylammonium; mono-, di- and tri-hydroxylalkylammonium; maleate; glutamate; borate; lactate; L-(+) ascorbic acid or salts thereof; reduced γ-glutathione; β-mercaptoethanol; thioglycerol; N-acetyl-L-cysteine, and combinations of these.

4. The concentrated aqueous solution according to claim 1 which contains at least 50 mg/ml of 5,10-methylene-(6R)-tetrahydrofolic acid.

5. A reconstituted product obtained by diluting the concentrated aqueous solution of claim 1 in water or a liquid pharmaceutically acceptable vehicle.

6. The reconstituted product according to claim 5, wherein the water is sterile water for injection.

7. The reconstituted product according to claim 5, further comprising a pharmaceutically acceptable carrier.

8. The reconstituted product according to claim 5, further comprising an additional pharmaceutically acceptable active ingredient.

9. The reconstituted product according to claim 5, further comprising a buffer, one or more osmolality correcting excipients, or both.

10. The reconstituted product according to claim 5, for use in the treatment of cancer or in cancer therapy.

11.-14. (canceled)
